(19) United States
(12) Patent Application Publication
(10) Pub. No.: US 2021/0261306 A1
(43) Pub. Date: Aug. 26, 2021

(54) LIGHT PROTECTION CLOSURE

(71) Applicant: THE CHEMOURS COMPANY FC, LLC, WILMINGTON, DE (US)

(72) Inventor: CHERYL MARIE STANCIK, KENNETT SQUARE, PA (US)

(73) Assignee: THE CHEMOURS COMPANY FC, LLC, WILMINGTON, DE (US)

(21) Appl. No.: 17/251,249

(22) PCT Filed: Jun. 11, 2019
(86) PCT No.: PCT/US2019/036507

§ 371 Date: Dec. 11, 2020

Related U.S. Application Data
(60) Provisional application No. 62/684,221, filed on Jun. 13, 2018.

(51) Int. Cl. B65D 51/24; B65D 81/30; G01N 17/00; G01N 33/04

(52) U.S. Cl. CPC B65D 51/245; G01N 33/04; G01N 17/004; B65D 81/30

(57) ABSTRACT

A new light protective closure that includes a wall portion(s) and a top plate. The top plate is provided with an at least partial supplemental light protection layer in addition to the light protection provided by the material used to form the top plate.

### BACKGROUND OF THE INVENTION

Certain compounds and nutrients contained within packages can be negatively impacted by exposure to light. Many different chemical and physical changes may be made to molecular species as a result of either a direct, or indirect, exposure to light, which can collectively be defined as photochemical processes. As described in Atkins, photochemical processes can include primary absorption, physical processes (e.g., fluorescence, collision-induced emission, stimulated emission, intersystem crossing, phosphorescence, internal conversion, singlet electronic energy transfer, energy pooling, triplet electronic energy transfer, triplet-triplet absorption), ionization (e.g., Penning ionization, dissociative ionization, collisional ionization, associative ionization), or chemical processes (e.g., disassociation or degradation, addition or insertion, abstraction or fragmentation, isomerization, dissociative excitation) (Atkins, P. W.; Table 26.1 Photochemical Processes. Physical Chemistry, 5th Edition; Freeman: New York, 1994; 908.). As one example, light can cause excitation of photosensitizer species (e.g., riboflavin in dairy food products) that can then subsequently react with other species present (e.g., oxygen, lipids) to induce changes, including degradation of valuable products (e.g., nutrients in food products) and evolution of species that can adjust the quality of the product (e.g., off-odors in food products).

As such, there is a need to provide packaging with sufficient light protection properties to allow the protection of the package content(s) and sufficient mechanical properties to withstand shipping, storage, and use conditions.

The ability of packages to protect substances they contain is highly dependent on the materials used to design and construct the package (reference: Food Packaging and Preservation; edited M. Mathlouthi, ISBN: 0-8342-1349-4; Aspen publication; Copyright 1994; Plastic Packaging Materials for Food; Barrier Function, Mass Transport, Quality Assurance and Legislation: ISBN 3-527-28868-6; edited by O. G Piringer; A. L. Baner; Wiley-vch Verlag GmBH, 2000, incorporated herein by reference). Preferred packaging materials are designed with consideration for the penetration of moisture, light, and oxygen often referred to as barrier characteristics.

Light barrier characteristics of materials used for packaging are desired to provide light protection to package contents. Methods have been described to measure light protection of a packaging material and characterize this protection with a “Light Protection Factor” or (LPF) as described in commonly owned U.S. Pat. No. 9,638,679 “Methods for producing new packaging designs based on photoprotective materials”, the subject matter which is hereby incorporated by reference in its entirety. See also, “Accelerated light protection performance measurement technology validated for dairy milk packaging design”, Stancik, Cheryl M.; Conner, Denise A.; Jernakoff, Peter; Niedenzu, Philipp M.; Duncan, Susan E.; Bianchi, Laurie M.; Johnson, Daryan S., Packaging Technology and Science, DOI 10.1002/pts.2326; June 2017.

Titanium dioxide (TiO₂) is frequently used in plastics food packaging layer(s) at low levels (typical levels of 0.1 weight % to 5 weight % (“weight %” is abbreviated as “wt %” hereinafter) of a composition) to provide aesthetic qualities to a food package such as whiteness and/or opacity. In addition to these qualities, titanium dioxide is recognized as a material that may provide light protection of certain entities as described in, for example, U.S. Pat. Nos. 5,750,226; 6,465,062; and US 2004/0195141.

Useful packaging designs are those that provide the required light protection and functional performance at a reasonable cost for the target application. The cost of a packaging design is in part determined by the materials of construction and the processing required to create the packaging design.

Milk packaging is an application where there is a benefit for light protection in packages to protect milk from the negative impacts of light exposure. Light exposure to milk may result in the degradation of some chemical species in the milk; this degradation results in a decrease in the nutrient levels and sensory quality of the milk (e.g., “Riboflavin Photosensitized Singlet Oxygen Oxidation of Vitamin D”, J. M. King and D. B. Min, V 63, No. 1, 1998, Journal of Food Science, page 31). Hence protection of milk from light with light protection packaging will allow the nutrient levels and sensory quality to be preserved at their initial levels for extended periods of time as compared to milk packaged in typical packaging that does not have light protection (e.g., “Effect of Package Light Transmittance on Vitamin Content of Milk. Part 2: UHT Whole Milk.” A. Saffert, G. Pieper, J. Jetten; Packaging Technology and Science, 2008; 21: 47-55).

State of the art packages fail to consider all portions of the light exposed bottle design including all areas of the package that allow the potential for light exposure to the product contained within. For example, for a light protection dairy bottle, this includes the bottle (all surfaces top, shoulders, sides, base) as well as the closure (e.g., cap). For optimal light protection performance, the bottle and cap should have substantially the same light protection performance or alternatively, when the light protection performance of the bottle and cap are different, the desired light protection performance should be met by the minimum performance level for either the bottle or cap.

Commonly owned PCT/US2018/025372 provides one solution to the above problem. Disclosed are novel light protection packages including a monolayer container and monolayer closure that considers all portions of the light exposed package design, including all areas of the package that allow the potential for light exposure to the product contained in the package. Specifically disclosed are monolayer closures comprising a top portion that is a sufficient thickness produced with light protection materials to provide light protection performance to the closure. For example, the monolayer closure top portion can have a thickness of at least about 50 mils.

The invention provides a solution to the above-described problem with the combination of a closure and a light protective film (e.g., a label) affixed to the light vulnerable portions of the closure, namely the portion of the closure top plate that covers the opening in the bottle. With correct identification and sizing of the light protective label material to match the vulnerable dimensions of the closure, the closure could be rendered light protective with the light protective film affixed.

### SUMMARY OF THE INVENTION

The invention comprises a light protection closure (e.g., a cap) that comprises side wall(s) portion and a top plate, wherein the top plate is provided with supplemental light protection layer, such as a film, label, printed ink layer, etc., in addition to the light protection provided by the material used to form the top plate. The closure can be combined with a corresponding container (e.g., bottle, etc.) to provide a light protection package. For optimal light protection performance, the container and top plate portion with supplemental light protection layer can have substantially the same light protection performance or alternatively, when the light protection performance of the container and top plate portion with supplemental light protection layer are different, the desired light protection performance should be met by the minimum performance level for either the container or top plate portion with supplemental light protection layer.

### BRIEF DESCRIPTION OF THE FIGURES

FIG. 1 shows in cross-section a closure.

FIG. 2 shows in cross-section a closure according to the invention.

FIG. 3a shows in cross-section a closure and container according to the present invention.

FIG. 3b shows a top view of a closure according to the present invention.

### DETAILED DESCRIPTION OF THE DISCLOSURE

The invention comprises a light protection closure (e.g., a cap) that comprises side wall(s) portion and a top plate, wherein the top plate is provided with a supplemental light protection layer in addition to the light protection provided by the material used to form the top plate. The closure can be combined with a corresponding container (e.g., bottle, etc.) to provide a light protection package. For optimal light protection performance, the container and top plate portion with supplemental light protection layer can have substantially the same light protection performance or alternatively, when the light protection performance of the container and top plate portion with supplemental light protection layer are different, the desired package light protection performance should be met by the minimum performance level for either the container or top plate portion with supplemental light protection layer.

As used herein “comprising” is to be interpreted as specifying the presence of the stated features, integers, steps, or components as referred to, but does not preclude the presence or addition of one or more features, integers, steps, or components, or groups thereof. Additionally, the term “comprising” is intended to include examples encompassed by the terms “consisting essentially of” and “consisting of.” Similarly, the term “consisting essentially of” is intended to include examples encompassed by the term “consisting of.”

As used herein, when an amount, concentration, or other value or parameter is given as either a range, typical range, or a list of upper typical values and lower typical values, this is to be understood as specifically disclosing all ranges formed from any pair of any upper range limit or typical value and any lower range limit or typical value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, terms in the singular and the singular forms “a,” “an,” and “the,” for example, includes plural references unless the content clearly dictates otherwise. Thus, for example, reference to “TiO₂ particle”, “a TiO₂ particle”, or “the TiO₂ particle” also includes a plurality of TiO₂ particles. All references cited in this patent application are herein incorporated by reference.

The closure can be removable and re-sealable (such as a bottle cap, which may comprise threads). In an aspect of the invention the closure comprises plastic. In a further aspect of the invention the plastic closure can be combined with a corresponding container to form a dairy product package, for example a milk container and closure.

The supplemental light protection layer can be any suitable material that, when combined with the closure top plate, will increase the light protection factor (“LPF”) value of the top plate, when compared to the top plate alone. The light protection layer material can be, for example, labels, films, ink layers, etc. Non-limiting examples of supplemental light protection layer materials include, metals, metal foils, metalized plastics, printed or pigmented plastic. The film material can be a label. The film can comprise a sticker. The supplemental light protection layer can be moisture resistant. The supplemental light protection layer can be temperature tolerant. The supplemental light protection layer can be a thin film, such as a metal or metalized foil. The supplemental light protection layer can also be a light reflective material.

The top plate of the closure is provided with one or more additional layers covering at least the portion of the top plate that will cover the opening in a corresponding container. Such layer or layers may be formed from a label, paper, printed ink, wrap, coating treatment or other material. The layer or layers may cover more than the portion of the top plate that will cover the opening in a corresponding container and may extend to the edge(s) of the top plate. The layer or layers may be on the inner surface of the top plate, the outer surface, or both. The layer or layers contribute additional light protection performance to the package. The layer may serve other functions as well such has improved sealing performance or providing branding information.

In an aspect of the invention, injection molding can be used to produce the closure.

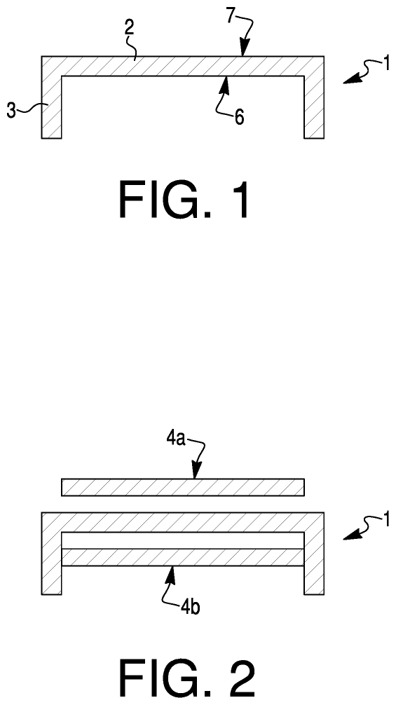

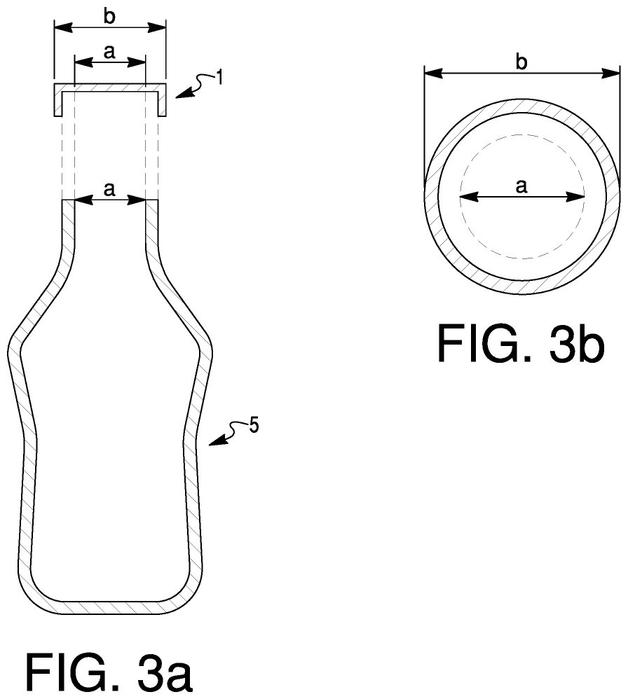

As shown in FIG. 1, the closure 1 comprises a top plate 2 and side wall(s) portion 3. Side wall(s) portion 3 can be any suitable geometric shape, but typically can be, for example, cylindrical or oval shaped, depending on the corresponding opening in the container. Although shown as a single wall construction, the wall portion may be fabricated in more than one section. As shown in FIG. 2, the closure top plate 2 is provided with a supplemental light protection layer(s) 4a, 4b to increase the light protection value across at least the portion of the top plate 2 that covers the opening a in the corresponding container 5, as shown in FIGS. 3a and 3b. The closure may be comprised of more than one material or layer and may contain layers for purposes like gas barrier or oxygen scavenging. The layer may be on the underside of the closure toward the enclosed product.

The supplemental light protection layer(s) 4a, 4b can be located on the inside surface 6 of the top plate 2, the outside surface 7, or both the inside 6 and outside 7 surfaces of the top plate 2. The supplemental light protection layer(s) 4a, 4b is of sufficient dimensions to at least cover the area of the top plate that will cover the opening a in container 5. The supplemental light protection layer(s) can be affixed to the top plate by any suitable means and may be removable or permanently affixed thereto.

The closure 1 can be used in conjunction with any container 5 (e.g., a bottle) wherein the closure 1 is designed to seal the opening a in the container 5. The closure can be removable and re-sealable and can be provided with threaded side wall(s) or side wall(s) otherwise designed to engage or seal with the corresponding container, such as a closure that can be sealed by pressing the cap to engage with the opening of the container to reseal the container.

Although the closure of the present invention can be used with any suitable container, preferred containers include the containers disclosed in commonly owned PCT/US2018/025372, US20160083554, WO2016/196529, and PCT patent application PCT/US2017/066105, the subject matter of each is hereby incorporated by reference in their entirety.

A detailed description of LPF and measuring LPF values is described in commonly owned U.S. Pat. No. 9,638,679 “Methods for producing new packaging designs based on photoprotective materials” and U.S. Pat. No. 9,372,145 “Devices for determining photoprotective materials” the subject matter of both patents is incorporated herein by reference. Additional information may be found in the example sections of these patents. LPF values used herein are determined according to the teachings in these two patents.

The closure (and container) can comprise plastic and can further comprise TiO₂ particles. Moreover, the closure (and container) can further comprise at least one color pigment. The TiO₂ particles and at least one color pigment can be dispersed throughout the closure (and container) material. The closure top plate with supplemental light protection layer can have a light protection factor (“LPF”) value of 20 or greater, preferably greater than 30, more preferably greater than 40, more preferably greater than 50, more preferably greater than 60, more preferably greater than 80, and even more preferably greater than 100. The container can have a light protection factor (“LPF”) value of 20 or greater, preferably greater than 30, more preferably greater than 40, more preferably greater than 50, more preferably greater than 60, more preferably greater than 80, and even more preferably greater than 100.

The titanium dioxide (and optionally at least one color pigment) can be present in the closure and be dispersed and processed in package production processes by incorporating a masterbatch, and preferably processed into a closure using injection molding. The masterbatch can be solid pellets. The masterbatch can be delivered as a liquid. The TiO₂ (and optional color pigment) could also be delivered in other forms, such as in a liquid delivery form and do not have to be delivered in one single masterbatch formulation.

In an aspect of the invention the TiO₂ particles can be coated with a metal oxide, preferable alumina, and then an additional organic layer. The treated TiO₂ is an inorganic particulate material that can be uniformly dispersed throughout a polymer melt, and imparts color and opacity to the polymer melt. Reference herein to TiO₂ without specifying additional treatments or surface layers does not imply that it cannot have such layers.

It is preferred that the metal oxide is selected from the group consisting of silica, alumina, zirconia, or combinations thereof. It is most preferred that the metal oxide is alumina. It is preferred that the organic coating material on the TiO₂ is selected from the group consisting of an organo-silane, an organo-siloxane, a fluoro-silane, an organo-phosphonate, an organo-acid phosphate, an organo-pyrophosphate, an organo-polyphosphate, an organo-metaphosphate, an organo-phosphinate, an organo-sulfonic compound, a hydrocarbon-based carboxylic acid, an associated ester of a hydrocarbon-based carboxylic acid, a derivative of a hydrocarbon-based carboxylic acid, a hydrocarbon-based amide, a low molecular weight hydrocarbon wax, a low molecular weight polyolefin, a co-polymer of a low molecular weight polyolefin, a hydrocarbon-based polyol, a derivative of a hydrocarbon-based polyol, an alkanolamine, a derivative of an alkanolamine, an organic dispersing agent, or a mixture thereof. It is more preferred that the organic material is an organo-silane having the formula: R⁵ₓSiR⁶₄₋ₓ wherein R⁵ is a nonhydrolyzable alkyl, cycloalkyl, aryl, or aralkyl group having at least 1 to about 20 carbon atoms; R⁶ is a hydrolyzable alkoxy, halogen, acetoxy, or hydroxy group; and x=1 to 3. It is most preferred that the organic material is Octyltriethoxysilane. In a further aspect of the invention the metal oxide is alumina and the organic material is octyltriethoxysilane.

In an aspect of the invention the closure can have a concentration of TiO₂ particles of from above 0 wt % to about 3 wt %. In a further aspect of the invention the closure can have a concentration of TiO₂ of less than 1 wt %, and may be less than 0.5 wt %. The melt processable resin(s) can be selected from the group of polyolefins. In an aspect of the invention the melt processable resin is preferably a high-density polyethylene and the closure can have a thickness of 8 mil to 50 mil, or more preferably 10 mil to 35 mil.

TiO₂ particles may be in the rutile or anatase crystalline form. It is commonly made by either a chloride process or a sulfate process. In the chloride process, TiCl₄ is oxidized to TiO₂ particles. In the sulfate process, sulfuric acid and ore containing titanium are dissolved, and the resulting solution goes through a series of precipitation steps to yield TiO₂. Both the sulfate and chloride processes are described in greater detail in “The Pigment Handbook”, Vol. 1, 2nd Ed., John Wiley & Sons, NY (1988), the teachings of which are incorporated herein by reference.

TiO₂ particles may have a medium diameter range of about 100 nm to about 250 nm as measured by X-Ray centrifuge technique, specifically utilizing a Brookhaven Industries model TF-3005W X-ray Centrifuge Particle Size Analyzer. The crystal phase of the TiO₂ is preferably rutile. The TiO₂ after receiving surface treatments can have a mean size distribution in diameter of about 100 nm to about 400 nm, more preferably about 100 nm to about 250 nm. Nanoparticles (those have mean size distribution less than about 100 nm in their diameter) could also be used in this invention but may provide different light protection performance properties.

The TiO₂ particles may be substantially pure, such as containing only titanium dioxide, or may be treated with other metal oxides, such as silica, alumina, and/or zirconia. TiO₂ particles coated/treated with alumina are preferred in the present invention. The TiO₂ particles may be treated with metal oxides, for example, by co-oxidizing or co-precipitating inorganic compounds with metal compounds. If a TiO₂ particle is co-oxidized or co-precipitated, then up to about 20 wt % of the other metal oxide, more typically, 0.5 to 5 wt %, most typically about 0.5 to about 1.5 wt % may be present, based on the total particle weight.

The treated titanium dioxide can be formed, for example, by the process comprising: (a) providing titanium dioxide particles having on the surface of said particles a substantially encapsulating layer comprising a pyrogenically-deposited metal oxide or precipitated inorganic oxides; (b) treating the particles with at least one organic surface treatment material selected from an organo-silane, an organo-siloxane, a fluoro-silane, an organo-phosphonate, an organo-acid phosphate, an organo-pyrophosphate, an organo-polyphosphate, an organo-metaphosphate, an organo-phosphinate, an organo-sulfonic compound, a hydrocarbon-based carboxylic acid, an associated ester of a hydrocarbon-based carboxylic acid, a derivative of a hydrocarbon-based carboxylic acid, a hydrocarbon-based amide, a low molecular weight hydrocarbon wax, a low molecular weight polyolefin, a co-polymer of a low molecular weight polyolefin, a hydrocarbon-based polyol, a derivative of a hydrocarbon-based polyol, an alkanolamine, a derivative of an alkanolamine, an organic dispersing agent, or a mixture thereof; and (c) optionally, repeating step (b).

An example of a method of treating or coating TiO₂ particles with amorphous alumina is taught in Example 1 of U.S. Pat. No. 4,460,655 incorporated herein by reference. In this process, fluoride ion, typically present at levels that range from about 0.05 wt % to 2 wt % (total particle basis), is used to disrupt the crystallinity of the alumina, typically present at levels that range from about 1 wt % to about 8 wt % (total particle basis), as the latter is being deposited onto the titanium dioxide particles. Note that other ions that possess an affinity for alumina such as, for example, citrate, phosphate or sulfate can be substituted in comparable amounts, either individually or in combination, for the fluoride ion in this process. The performance properties of white pigments comprising TiO₂ particles coated with alumina or alumina-silica having fluoride compound or fluoride ions associated with them are enhanced when the coated TiO₂ is treated with an organosilicon compound. The resulting compositions are particularly useful in plastics applications. Further methods of treating or coating particles of the present invention are disclosed, for example, in U.S. Pat. No. 5,562,990 and US 2005/0239921, the subject matter of which is herein incorporated by reference.

Titanium dioxide particles may be treated with an organic compound such as low molecular weight polyols, organosiloxanes, organosilanes, alkylcarboxylic acids, alkylsulfonates, organophosphates, organophosphonates and mixtures thereof. The preferred organic compound is selected from the group consisting of low molecular weight polyols, organosiloxanes, organosilanes and organophosphonates and mixtures thereof and the organic compound is present at a loading of between 0.2 wt % and 2 wt %, 0.3 wt % and 1 wt %, or 0.7 wt % and 1.3 wt % on a total particle basis. The organic compound can be in the range of about 0.1 to about 25 wt %, or 0.1 to about 10 wt %, or about 0.3 to about 5 wt %, or about 0.7 to about 2 wt %. One of the preferred organic compounds used in the present invention is polydimethyl siloxane; other preferred organic compounds used in the present invention include carboxylic acid containing material, a polyalcohol, an amide, an amine, a silicon compound, another metal oxide, or combinations of two or more thereof.

In a preferred embodiment, the at least one organic surface treatment material is an organo-silane having the formula: R⁵ₓSiR⁶₄₋ₓ wherein R⁵ is a non hydrolyzable alkyl, cycloalkyl, aryl, or aralkyl group having at least 1 to about 20 carbon atoms; R⁶ is a hydrolyzable alkoxy, halogen, acetoxy, or hydroxy group; and x=1 to 3. Octyltriethoxysilane is a preferred organo-silane.

The following TiO₂ pigments may be useful TiO₂ particles in the present invention: Chemours Ti-Pure™ R-101, R-104, R-105, R-108, R-350, TS-1600, and TS-1601. Other TiO₂ grades with similar size and surface treatments may also be useful in the invention.

When the TiO₂ particles and color pigments are used in a polymer composition/melt, the melt-processable polymer that can be employed together with the TiO₂ particles and color pigments comprise a high molecular weight polymer, preferably thermoplastic resin. By “high molecular weight” it is meant to describe polymers having a melt index value of 0.01 to 50, typically from 2 to 10 as measured by ASTM method D1238-98. By “melt-processable,” it is meant a polymer must be melted (or be in a molten state) before it can be extruded or otherwise converted into shaped articles, including films and objects having from one to three dimensions. Also, it is meant that a polymer can be repeatedly manipulated in a processing step that involves obtaining the polymer in the molten state.

Suitable polymers include, by way of example but not limited thereto, polymers of ethylenically unsaturated monomers including olefins such as polyethylene, polypropylene, polybutylene, and copolymers of ethylene with higher olefins such as alpha olefins containing 4 to 10 carbon atoms or vinyl acetate; vinyls such as polyvinyl chloride, polyvinyl esters such as polyvinyl acetate, polystyrene, acrylic homopolymers and copolymers; phenolics; alkyds; amino resins; polyamides; phenoxy resins, polysulfones; polycarbonates; polyesters and chlorinated polyesters; polyethers; acetal resins; polyimides; and polyoxyethylenes. Mixtures of polymers are also contemplated. Polymers suitable for use in the present invention also include various rubbers and/or elastomers, either natural or synthetic polymers based on copolymerization, grafting, or physical blending of various diene monomers with the above-mentioned polymers, all as generally known in the art. Typically, the polymer may be selected from the group consisting of polyolefin, polyvinyl chloride, polyamide and polyester, and mixture of these. More typically used polymers are polyolefins. Most typically used polymers are polyolefins selected from the group consisting of polyethylene, polypropylene, and mixture thereof. A typical polyethylene polymer is low density polyethylene (LDPE), linear low density polyethylene (LLDPE), and high density polyethylene (HDPE). Additional polymers include, for example, polyethylene Terephthalate (PET, PETE), polypropylene (PP), polystyrene (PS), and polyvinyl chloride (PVC, vinyl).

A wide variety of additives may be present in the closure of this invention as necessary, desirable, or conventional. Such additives include polymer processing aids such as fluoropolymers, fluoroelastomers, etc., catalysts, initiators, antioxidants (e.g., hindered phenol such as butylated hydroxytoluene), blowing agent, ultraviolet light stabilizers (e.g., hindered amine light stabilizers or “HALS”), organic pigments including tinctorial pigments, plasticizers, antiblocking agents (e.g. clay, talc, calcium carbonate, silica, silicone oil, and the like) leveling agents, flame retardants, anti-cratering additives, and the like. Additional additives further include plasticizers, optical brighteners, adhesion promoters, stabilizers (e.g., hydrolytic stabilizers, radiation stabilizers, thermal stabilizers, and ultraviolet (UV) light stabilizers), antioxidants, ultraviolet ray absorbers, anti-static agents, colorants, dyes or pigments, delustrants, fillers, fire-retardants, lubricants, reinforcing agents (e.g., glass fiber and flakes), processing aids, anti-slip agents, slip agents (e.g., talc, anti-block agents), and other additives.

Any melt compounding techniques known to those skilled in the art may be used to process the compositions of the present invention. Closures of the present invention may be made after the formation of a masterbatch. The term masterbatch is used herein to describe a mixture of TiO₂ particles and color pigments (collectively called solids) which can be melt processed at high solids to resin loadings (generally 50-80 wt % by weight of the total masterbatch) in high shear compounding machinery such as Banbury mixers, continuous mixers or twin screw mixers, which are capable of providing enough shear to fully incorporate and disperse the solids into the melt processable resin. The resultant melt processable resin product is commonly known as a masterbatch, and is typically subsequently diluted or “letdown” by incorporation of additional virgin melt processable resin in plastic production processes. The letdown procedure is accomplished in the desired processing machinery utilized to make the final consumer article, whether it is sheet, film, bottle, package or another shape. The amount of virgin resin utilized and the final solids content is determined by the use specifications of the final consumer article. The masterbatch composition of this invention is useful in the production of shaped articles.

In another embodiment of the present invention, the titanium dioxide and color pigment are supplied for processing into the closures as a masterbatch concentrate. Preferred masterbatch concentrates typically have titanium dioxide content of greater than 40 wt %, greater than 50 wt %, greater than 60 wt %, greater than 70 wt %, or greater than 80 wt %. Preferred color concentrate masterbatches are solid. Liquid color concentrates and/or a combination of liquid and solid color concentrates could be used.

In an aspect of the invention, the amount of titanium dioxide particles in the closure of the invention can be any suitable amount which results in the desired LPF value. For example, the amount of titanium dioxide particles contained in the container and/or closure can be at least about 0.5 wt %, and preferably at least about 0.1 wt %. In an aspect of the invention the titanium dioxide particles in the container and/or closure can be from about 0.5 wt % to about 20 wt %, and is preferably from about 0.1 wt % to about 15 wt %, more preferably 5 wt % to 10 wt %. In a further aspect of the invention the titanium dioxide particles in the container and/or closure can be from at least about 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt % to 12 wt %. In an aspect of the invention the titanium dioxide particles in the container and/or closure can be any amount between 0.1 wt % and 12 wt % (all wt % are based on the total weight of the closure without considering the supplemental light protection layer.

A closure is typically produced by melt blending the masterbatch containing the titanium dioxide and color pigment with a second high molecular weight melt-processable polymer to produce the desired composition used to form the finished closure. The masterbatch composition and second high molecular weight polymer can be melt blended, using any means known in the art, as disclosed above in desired ratios to produce the desired composition of the final closure. In this process, twin-screw extruders are commonly used. The resultant melt blended polymer is extruded or otherwise processed to form a closure of the desired composition, for example by injection molding processing.

The closure can be combined with a corresponding container to form a package. The package finds utility to contain dairy and non-dairy milk products, usually liquids. Liquid should be understood to mean a liquid that is taken or derived from a protein source, such as coconut, soybean, cows, goats, etc. Non-dairy milk includes, for example, liquid derived from almonds, cashews, coconuts, flax, soy, rice, hazelnut, hemp, quinoa, etc.

Measuring Light Protection Performance or LPF

The LPF value quantifies the protection a packaging material can provide for a light sensitive entity in a product when the packaged product is exposed to light. The LPF value for a packaging material is quantified in our experiment as the time when half of the product light sensitive entity concentration has been degraded or otherwise undergone transformation in the controlled experimental light exposure conditions. Hence, a product comprising one or more light sensitive entities protected by a high LPF value package can be exposed to a larger dose of light before changes will occur to the light sensitive entity versus the product protected by a low LPF value package.

A detailed description of measuring LPF value is further described in commonly owned U.S. Pat. No. 9,638,679 titled, “Methods for Determining Photo Protective Materials” and U.S. Pat. No. 9,372,145 titled, “Devices for Determining Photo Protective Materials incorporated herein by reference.

### EXAMPLES

Applying the teachings of commonly owned PCT/US2018/025372, US2018/0134875, U.S. Pat. Nos. 9,372,145, and 9,638,679, dairy package closures were evaluated for their light protection factor (“LPF”) values.

As disclosed in US2018/0134875, the light protection performance of a packaging material can be quantified with a light protection factor (“LPF”) value. Further, as disclosed in U.S. Pat. Nos. 9,372,145 and 9,638,679 sample holders can be selected to hold different types of packaging samples. As disclosed in PCT/US2018/025372, a specialized sample holder for evaluating caps was designed and characterized. Data collected here using the cap sample holder was normalized back to the standard LPF scales using the approaches described in the Examples of PCT/US2018/025372.

With this approach, the following data was collected.

A set of identical white plastic closures typical of those used for dairy milk applications was obtained from retail. The closure was typical of those used in milk applications.

The white plastic closure obtained from retail (Sample A) was measured for LPF performance using the method and cap sample holder disclosed in PCT/US2018/025372 and then normalized back to the standard LPF scale. All data in this example are reported on the standard scale.

For a preferred light protection package closure design of the invention, an LPF of greater than 40 is preferred.

The measured LPF value of 5 of the white cap closure (sample A) is insufficient to produce the required light protection performance.

A paper label commercially available from Avery (#5896), having a thickness of 4.0 mil and an integrated adhesive side, was cut to a diameter of about 37 mm and applied to a white cap sample over the entire top plate to form Sample B. Sample B was measured essentially the same as Sample A and found to provide some benefit with an improved LPF value of 24, this value is still insufficient to produce the desired light protection performance of LPF greater than 40.

A foil label (Metalized Silver Permanent label obtained from Label Value, Tampa, Fla., having a thickness of 3.5 mil and an integrated adhesive side) having a diameter of about 37 mil was applied to a white cap sample to cover the entire top plate portion of the cap to form Sample C. Sample C was measured essentially the same as Samples A and B and found to have an improved LPF value of greater than 100. This data indicates this design is sufficient to produce the desired light protection performance of LPF greater than 40.

By using the label that is both the correct material and sufficiently sized for the cap structure completely covering the cap top plate portion above the bottle opening, the LPF performance of greater than 100 is achieved thus meeting the desired light protection performance.

Thus, it is demonstrated that by selecting an appropriate light protection label with the correct dimensions of the cap requiring additional light protection, that at closure can be rendered with appropriate light protection performance.

What is claimed is:

1. A closure comprising side wall(s) portion and a top plate wherein the top plate is at least partially covered with supplemental light protection layer and wherein the top plate and the supplemental light protection layer combined provide a LPF value greater than 25.

2. The closure of claim 1 wherein the LPF is greater than 50.

3. The closure of claim 1 wherein the LPF is greater than 100.

4. The closure of claim 1 wherein the closure can be removed and reapplied to a corresponding container.

5. The closure of claim 1 wherein the supplemental light protection layer comprises a label material.

6. The closure of claim 5 wherein the label comprises a sticker.

7. The closure of claim 6 wherein the sticker is moisture resistant.

8. The closure of claim 6 wherein the sticker comprises a thin film.

9. The closure of claim 8 wherein the thin film comprises metal foil.

10. The closure of claim 1 wherein the supplemental light protection layer is provided to any of a top surface of the closure or a bottom surface of the top plate.

11. The closure of claim 6 wherein the sticker comprises a material that is light reflective.

12. The closure of claim 6 wherein the sticker comprises at least two different colors.

13. The closure of claim 1 wherein the closure can comprise polyethylene, polypropylene, or combinations thereof.

14. The closure of claim 1 comprising colored pigment.

15. The closure of claim 1 comprising white pigment.

16. The closure of claim 1 comprising additives.

17. The closure of claim 1 wherein the closure is combined with a container to form a package.

18. The closure of claim 17 wherein the container and the closure have substantially the same LPF value.

19. The closure of claim 18 wherein the container has an LPF greater than 25.

20. The closure of claim 18 wherein the container has an LPF greater than 50.

21. The closure of claim 18 wherein the container has an LPF greater than 75.

22. The closure of claim 18 wherein the container has an LPF greater than 100.

23. The closure of claim 17 wherein the package contains dairy product.

24. The closure of claim 1 wherein the supplemental light protection layer comprises a film material.

25. The closure of claim 1 wherein the supplemental light protection layer comprises a printed ink layer.

26. The closure of claim 1 wherein the supplemental light protection layer remains in contact with the closure when the closure is removed from a corresponding container.
